(19) United States
(12) Patent Application Publication
(10) Pub. No.: US 2003/0236539 A1
(43) Pub. Date: Dec. 25, 2003

(54) Apparatus and method for using an ultrasonic probe to clear a vascular access device

(75) Inventors: Robert A. Rabiner, North Reading, MA (US); Bradley A. Hare, Chelmsford, MA (US); Rebecca I. Marciante, North Reading, MA (US); Kevin J. Ranucci, North Attleboro, MA (US); Mark J. Varady, Holliston, MA (US); Roy M. Robertson, Ipswich, MA (US); Janniah S. Prasad, Norwalk, CT (US); Scott A. Talbot, North Andover, MA (US); Peter C. Colgan, Bedford, MA (US)

Correspondence Address: PALMER & DODGE, LLP, RICHARD B. SMITH, 111 HUNTINGTON AVENUE, BOSTON, MA, 02199, US

(73) Assignee: OmniSonics Medical Technologies, Inc.

(21) Appl. No.: 10/396,923

(22) Filed: Mar. 25, 2003

Related U.S. Application Data
(63) Continuation-in-part of application No. 09/776,015, filed on Feb. 2, 2001.
Application No. 09/776,015 is a continuation-in-part of application No. 09/618,352, filed on Jul. 19, 2000, now Pat. No. 6,551,337.
(60) Provisional application No. 60/178,901, filed on Jan. 28, 2000.
(60) Provisional application No. 60/157,824, filed on Oct. 5, 1999.

(51) Int. Cl. A61B 17/22; A61D 1/02

(52) U.S. Cl. 606/159

(57) ABSTRACT

The present invention provides an apparatus and a method for using an ultrasonic probe to remove an occlusion in vascular access devices including fistulas, grafts, catheters and subcutaneous access devices. The ultrasonic probe is inserted into the vascular access device and a section of a longitudinal axis of the ultrasonic probe engages the occlusion. A transducer transmits an ultrasonic energy from an ultrasonic energy source that produces a transverse ultrasonic vibration along the longitudinal axis of the ultrasonic probe. The transverse ultrasonic vibration of the ultrasonic probe provides a plurality of transverse anti-nodes along a portion of the longitudinal axis of the ultrasonic probe that cause a cavitation in a medium in communication with the ultrasonic probe to ablate the occlusion.

### RELATED APPLICATIONS

This application is a continuation-in-part of Application Serial Number 09/776,015, filed Feb. 2, 2001, which is a continuation-in-part of Application Serial No. 09/618,352, filed Jul. 19, 2000, which claims benefit of Provisional Application Serial No. 60/178,901, filed Jan. 28, 2000, and claims benefit of Provisional Application Serial No. 60/157,824, filed Oct. 5, 1999, the entirety of all these applications are hereby incorporated herein by reference.

### FIELD OF THE INVENTION

The present invention relates to an ultrasonic medical device, and more particularly to an apparatus and a method of using an ultrasonic probe to clear an occlusion in a vascular access device to keep the vascular access device clear of the occlusion and prevent subsequent health risks.

### BACKGROUND OF THE INVENTION

The use of vascular access devices has become a common practice across the world to address various health issues. Vascular access devices are used to administer pharmacological agents and to draw blood from vasculatures within the body. There are several different types of vascular access devices, with the choice of the vascular access device depending upon the type of treatment that is needed, the amount of time the patient will need the vascular access device, the type of pharmacological agent the patient needs and the condition of the patient's veins. Some patients require temporary vascular access devices while others require permanent vascular access devices. The use of vascular access devices has become especially important in cystic fibrosis patients who require frequent and prolonged intravenous antibiotics. Vascular access devices are also used in hemodialysis patients who require a treatment of the blood.

Major health issues arise as a result of the improper functioning of the kidneys. Healthy humans have two kidneys, each about the size of an adult fist, located on either side of the spine just below the rib cage. Although the kidneys are small, the kidneys perform many complex and vital functions that keep the rest of the body in balance. For example, kidneys help remove waste and excess fluid, filter the blood (keeping some compounds while removing others), control the production of red blood cells, release hormones that help regulate blood pressure, make vitamins that control growth, and help regulate blood pressure, red blood cells, and the amount of certain nutrients in the body, such as calcium and potassium.

Kidneys that are not functioning effectively require a procedure called dialysis, a process of removing waste products and excess fluid which build up in the body when the kidneys are not functioning well. Dialysis is necessary when a patient's kidneys can no longer take care of the patient's bodily needs. Dialysis is a medical procedure routinely used in end-stage renal disease (ESRD), also known as end stage kidney failure, usually by the time the patient has lost about 85 to 90 percent of kidney function. Adequate care of an ESRD hemodialysis dependent patient requires constant attention to the need to maintain vascular access patency. Dialysis is a standard treatment of ESRD all around the world, with thousands of patients being helped by dialysis treatment.

Like healthy kidneys, dialysis keeps the patient's body in balance by removing waste, salt and extra water to prevent them from building up in the body, keeping a safe level of certain chemicals in the patient's blood, such as potassium, sodium and bicarbonate, and helping to control blood pressure. Dialysis uses a membrane as a filter and a solution called dialysate to regulate the balance of fluid, salts and minerals carried in the bloodstream. The membrane may be man-made as in hemodialysis or natural as in peritoneal dialysis.

Hemodialysis is a medical procedure used routinely in the treatment of end-stage renal disease, in which the patient's blood is shunted from the body through a hemodialyser for diffusion and ultrafiltration, and then returned to the patient's vascular system. Hemodialysis removes certain elements from the blood by virtue of the difference in the rates of their diffusion through a semipermeable membrane, for example, by means of a hemodialysis machine or a filter. In hemodialysis, a hemodialyser (commonly referred to as an artificial kidney) is used to clean a patient's blood by removing waste and extra chemicals and fluid from the patient's blood. A hemodialyser works on the principle of blood flowing along one side of a semi-permeable cellulose membrane or a similar product, while the dialysate flows along the other side. The dialysate contains a regulated amount of minerals normally present in the blood, but in renal failure they are present in excess. The membrane has tiny holes of different sizes so that the excess fluid and substances in the blood pass through at different rates, small molecules quickly and larger ones more slowly, to be taken away in the dialysate until a correct balance in the blood is achieved.

During hemodialysis, a kidney machine regulates blood flow, pressure and the rate of exchange. As only a very small amount of blood is in the hemodialyser at any given time, blood needs to circulate from patient to hemodialyser and back to patient for approximately four hours. Hemodialysis treatments typically occur three times per week, with the time and strength of hemodialysis programmed for each patient.

In order to be able to get a patient's blood for use in a procedure such as hemodialysis, there must be an access (entrance) into the patient's blood vessels. A vascular access device is a way to reach the blood for use in the particular procedure. An ideal vascular access device delivers a flow rate adequate for the dialysis prescription, has a long use-life and has a low rate of complications including infection, stenosis, thrombosis, aneurysm and limb ischemia. There are four common types of vascular access devices: (1) an arterivenous (“AV”) fistula; (2) an AV graft; (3) a catheter; and (4) a subcutaneous access device. Such vascular access is usually accomplished by minor surgery to a patient.

AV fistulas are formed internally by a surgical anastomosis joining an artery to a vein under the patient's skin, usually in the forearm or wrist, to allow for arterial blood flow directly into the vein. Fistulas are a permanent access that have been a preferred vascular access device for long term dialysis patients. The use of a fistula for a patient is dependent upon the size of the patient's veins and the amount of time available to create the fistula. Fistulas should be placed several months prior to the initiation of hemodialysis to allow for proper healing before use. Two to three months after the fistula is surgically formed, the fistula matures creating a larger blood vessel with strong walls and easier, less painful vascular access. The subsequent increase in flow of arterial blood into the vein permits percutaneous puncture of the blood vessel, allowing needles to be inserted and removed during each hemodialysis treatment. Between hemodialysis treatments, only a small scar and swelling are visible on the patient.

Although fistulas can last for years, there is a risk of infection and stenosis or narrowing of the fistula. Once the fistula becomes occluded, vascular access may be lost requiring placement of either a fistula or a graft in another location. Pharmacological agents that treat blood clots may be used to reverse stenosis of the fistula, however, these medications can cause complications including bleeding disorders, severe allergic reactions and death. When a fistula fails, or the patient's blood vessels are too small to create and maintain a fistula, AV grafts may be used for vascular access.

AV grafts are a reasonable alternative to fistulas, but grafts are not without problems. Grafts are formed by using either an artificial blood vessel or a larger vessel from the patient's own body to internally join an artery and a vein under the patient's skin, usually in the forearm or thigh. The graft is surgically placed close to the surface of the skin and may be utilized within two to four weeks after placement and provide for easier, less painful vascular access.

Grafts, as compared to fistulas, require shorter times to heal before they can be used, but grafts also have problems. Grafts usually do not last as long as fistulas and grafts have greater incidence of stenosis and thrombosis than fistulas. Because grafts are usually artificial and not a vessel obtained from the patient, infection, thrombosis, pseudoaneurysm, hematoma, and stenosis or narrowing of the graft may occur. If any of these complications do arise, vascular access may be lost. To prevent loss of vascular access, the graft must somehow be cleared. Currently, either clot-busting drugs that treat blood clots or surgery are available treatments. However, these treatments can be very invasive and do not come without risks including bleeding, allergic reactions, pulmonary embolism, cardiac arrest and death. The most frequently used graft is a synthetic graft made from polytetrafluoroethylene.

Catheters provide an access made by means of a flexible, hollow tube which is inserted into a large vein, usually in the patient's neck. Catheters, commonly referred to as temporary vascular access devices, are most often used as “bridge” devices, used to span the time between the commencement of dialysis treatments (often an emergency) to when the patient's AV fistula or AV graft has matured and is ready for use. Catheters are generally not used as long-term devices as they tend to have higher rates of infection and thrombosis.

There are several types of catheters that are used in procedures involving the exchange of blood. Internal jugular catheters are placed into the jugular vein on the side of the neck. Subclavian catheters are inserted into the subclavian vein under the collarbone on the chest. Femoral catheters are inserted into the large femoral vein in the leg close to the groin. Cuffed tunneled catheters, including silastic cuffed catheters, are designed to be placed under the skin and include an internal cuff to keep them in place. Cuffed tunneled catheters may be used for several months. Other types of catheters known in the art include non-cuffed catheters, peripherally inserted central catheters, apheresis catheters and triple lumen central venous catheters.

In response to the problems associated with vascular access by fistulas, grafts and catheters, subcutaneous access has been developed in which a vascular access device is implanted underneath the skin. One such subcutaneous access device comprises one or more small metallic devices implanted underneath the skin, usually in the upper chest. Since the subcutaneous access device is underneath the skin, the skin acts as a barrier to bacteria that can adversely affect the device and cause an infection. The small metallic devices are connected to two flexible tubes that are inserted into a large vein for blood access. The subcutaneous access devices have internal mechanisms that open upon introduction of a needle and close upon exit of the needle. Implantation of the metallic devices is a minor surgical procedure that allows the devices to be used on the same day as the surgical procedure. Subcutaneous access devices have shown the ability to provide high blood flows, decreased clotting and decreased rates of infection when compared to catheter access devices. A port is another type of subcutaneous access device.

For an exchange of blood procedure such as a hemodialysis treatment, if the patient's access is a fistula or a graft, the patient's nurse or technician will place two needles into the access at the beginning of each treatment. These needles are connected to dialysis lines (soft plastic tubes) that connect to the hemodialyser. Blood goes to the hemodialyser through one of the dialysis lines, gets cleaned in the hemodialyser, and returns to the patient through the other dialysis lines. If the patient's access is a catheter, the dialysis lines can be connected directly to the catheter without the use of needles. Subcutaneous access devices require the use of one needle.

Proper maintenance of the vascular access is as important as creating a quality vascular access. Whether the access is a fistula, graft, catheter or subcutaneous access device, the proper care for the vascular access device must be maintained so problems do not develop. The most common problems associated with vascular access include stenosis (narrowing of blood vessel/graft), occlusion formation (thrombosis and clotting), and infection.

Venous stenosis is the narrowing of the blood vessel or graft. Physiologically, venous stenosis increases resistance to blood flow, which in turn results in increased venous pressure, decreased blood flow and, ultimately, thrombosis. Moreover, the presence of venous stenosis reduces the efficiency of the hemodialysis treatment. Stenosis can and should be detected prospectively to allow swift, successful treatment. Correction of venous stenoses of greater than fifty percent lumen diameter can result in a significant decrease in the rate of fistula thrombosis and an improvement in access patency. Currently, stenosis is diagnosed by measuring the venous pressure at constant blood flow (200 ml/min) through the hemodialyser. Venous stenosis increases the risk of thrombosis.

Thrombosis is an obstruction of a blood vessel by a clot of coagulated blood formed at the site of obstruction. A thrombus is an aggregation of blood factors, primarily platelets and fibrin with entrapment of cellular elements, frequently causing vascular obstruction at the point of its formation. A thrombus is distinguished from an embolism, in that the embolism is produced by a clot or foreign body brought from a distance. Thrombosis results in an elevation of resistance and impairment of access flow.

Venous stenosis, occlusions and thrombotic episodes cause the vast majority of access failures in patients. Additionally, infection or other complications can also result in access failure. The complications of vascular access are not only a major cause of morbidity in hemodialysis patients, but a major cost for the end-stage renal disease treatment program. Access salvage includes prospective monitoring and treatment of outflow stenosis. The direct intra-access measure of blood flow by ultrasound dilution and a duplex color flow Doppler technique is the ideal method for detecting venous outflow stenosis. However, conventional and digital subtraction angiography has an advantage in that the total vascular system and blood flow may be visualized. The various treatment modalities for outflow stenosis include use of percutaneous transluminal angioplasty, stents, and surgical correction.

The prior art has not solved the problems of preventing occlusion formation in a vascular access device and removing an occlusion from the vascular access device. U.S. Pat. No. 5,464,438 to Menaker discloses an implantable graft lined or coated with gold to form a non-thrombogenic surface. Gold is sputtered onto the graft to allow contact between the gold and the blood. In addition to complexities with the administering of gold to a device, it is difficult to maintain the coated surface without the coating being removed and adversely affecting areas downstream of the coated graft. Since grafts undergo a lot of wear and tear, the gold coated graft of the Menaker device would not provide adequate long term viability. The use of gold is also an expensive approach in trying to provide an anti-thrombosis solution. Therefore, there remains a need in the art for a method of clearing a vascular access device that is simple, does not harm the vascular access device or the patient, does not adversely affect blood flow downstream of the vascular access device and effectively removes occlusions in vascular access devices.

U.S. Pat. No. 6,113,570 to Siegel et al. discloses the use of a combination of an echo contrast agent and ultrasonic energy applied to the exterior of the body proximate a thrombus to remove the thrombus residing in a fistulae. In the Siegel et al. device, an echo contrast agent and/or a thrombolytic agent are injected proximate a thrombus in a fistulae and ultrasound energy is applied transcutaneously with enough energy to increase the thrombolytic action of the thrombolytic agent and generate microbubbles in the echo contrast agent to clear the thrombus. Ultrasonic energy is applied by a transducer on the body and transmitted through the body, where it is subsequently dampened by the various layers between the transducer and the thrombus. The Siegel et al. device is not effective at removing a thrombus in a fistulae because the ultrasonic energy is not focused to generate direct and controlled motion of the microbubbles to effectively remove the thrombus. The use of a thrombolytic agent can result in adverse complications such as bleeding. Therefore, there remains a need in the art for a method of clearing a vascular access device that is simple, does not harm the vascular access device or the patient, does not adversely affect blood flow downstream of the vascular access device and effectively removes occlusions in vascular access devices.

All prior art treatments of removing occlusions in a vascular access device to preserve vascular access are complicated, invasive, expensive, not effective and subject the patient to minor and/or severe complications. Therefore, there is a continuing need in the art for further developments in the treatment of thrombosis to remove biological material from vascular access devices with minimal invasiveness and minimal risk to the patient. In particular, an apparatus and a method of utilizing an ultrasonic probe to remove an occlusion from a vascular access device in a patient with minimal invasiveness and minimal risk to the patient would further advance the state of the art.

### SUMMARY OF THE INVENTION

The present invention relates to an ultrasonic medical device, and more particularly to an apparatus and a method of using an ultrasonic probe to clear an occlusion in a vascular access device to keep the vascular access device clear of the occlusion and prevent subsequent health risks.

The present invention is an ultrasonic medical device comprising an ultrasonic probe and an ultrasonic energy source. A transducer having a first end engaging the ultrasonic energy source and a second end engaging a proximal end of the ultrasonic probe transmits an ultrasonic energy to the ultrasonic probe. The ultrasonic energy source produces a transverse ultrasonic vibration along a longitudinal axis of the ultrasonic probe to ablate an occlusion in a vascular access device. The vascular access device can be a fistula, a graft, a catheter or a subcutaneous access device.

The present invention is an elongated flexible probe for removing an occlusion in a vascular access device. The elongated flexible probe can support a transverse ultrasonic vibration along a portion of a longitudinal axis of the elongated flexible probe to remove the occlusion from the vascular access device.

The present invention provides a method of removing an occlusion from a vascular access device by inserting an ultrasonic probe into the vascular access device and activating an ultrasonic energy source. The ultrasonic energy source produces an ultrasonic energy that vibrates the ultrasonic probe in a transverse direction to ablate the occlusion in the vascular access device. The transverse ultrasonic vibration of the ultrasonic probe provides a plurality of transverse nodes and transverse anti-nodes along a portion of the longitudinal axis of the ultrasonic probe, causing a cavitation in a medium in communication with the ultrasonic probe to ablate the occlusion.

The present invention provides a method of ablating an occlusion in a vascular access device comprising inserting a segment of a longitudinal axis of an ultrasonic probe into the vascular access device, activating an ultrasonic energy source to produce a transverse ultrasonic vibration along the longitudinal axis of the ultrasonic probe and moving the segment of the longitudinal axis of the ultrasonic probe within the vascular access device to ablate the occlusion. A section of the longitudinal axis of the ultrasonic probe engages the occlusion and the occlusion is removed. The ultrasonic probe may be rotated, moved back and forth or swept along the occlusion within the vascular access device.

The present invention is an apparatus and a method using an ultrasonic probe to clear a vascular access device. The occlusion is removed by a cavitation produced by transverse antinodes along a portion of a longitudinal axis of the ultrasonic probe, produced from a transverse ultrasonic vibration of the ultrasonic probe. The present invention provides a method of effectively removing the occlusion from the vascular access device that is simple, user-friendly, effective, reliable and cost effective.

### BRIEF DESCRIPTION OF THE DRAWINGS

The present invention will be further explained with reference to the attached drawings, wherein like structures are referred to by like numerals throughout the several views. The drawings shown are not necessarily to scale, with emphasis instead generally being placed upon illustrating the principles of the present invention.

FIG. 1 shows a side plan view of an ultrasonic medical device of the present invention capable of operating in a transverse mode.

FIG. 2 shows an AV fistula formed by engaging an artery to a vein in an arm of a patient.

FIG. 3 shows a graft formed by engaging an artificial blood vessel to an artery on one end of the artificial blood vessel and a vein on the other end of the artificial blood vessel.

FIG. 4 shows a catheter inserted into a vein in the chest of a patient.

FIG. 5 shows a subcutaneous access device comprising a plurality of metallic devices engaging a vein in the chest of a patient.

FIG. 6 shows a side plan view of an ultrasonic probe with a plurality of transverse nodes and transverse anti-nodes along a portion of a longitudinal axis of the ultrasonic probe.

FIG. 7 shows a segment of a longitudinal axis of an ultrasonic probe inserted into a vascular access device and a section of the longitudinal axis of the ultrasonic probe engaging an occlusion in the vascular access device.

FIG. 8 shows a segment of a longitudinal axis of an ultrasonic probe inserted into a vascular access device with a section of the longitudinal axis of the ultrasonic probe engaging an occlusion that is partially removed.

FIG. 9 shows a segment of a longitudinal axis of an ultrasonic probe inserted into a vascular access device and a section of the longitudinal axis of the ultrasonic probe engaging an occlusion that is almost completely removed.

FIG. 10 shows a segment of a longitudinal axis of an ultrasonic probe inserted into a vascular access device in which the occlusion has been removed.

While the above-identified drawings set forth preferred embodiments of the present invention, other embodiments of the present invention are also contemplated, as noted in the discussion. This disclosure presents illustrative embodiments of the present invention by way of representation and not limitation. Numerous other modifications and embodiments can be devised by those skilled in the art which fall within the scope and spirit of the principles of the present invention.

### DETAILED DESCRIPTION

The present invention provides an apparatus and a method for using an ultrasonic medical device comprising an ultrasonic probe to ablate an occlusion in a vascular access device. Vascular access devices include, but are not limited to, fistulas, grafts, catheters, subcutaneous access devices and other similar devices. A segment of a longitudinal axis of the ultrasonic probe is inserted into the vascular access device and a section of the longitudinal axis of the ultrasonic probe engages the occlusion. A transducer having a first end engaging the ultrasonic energy source and a second end engaging a proximal end of the ultrasonic probe transmits an ultrasonic energy to the ultrasonic probe when the ultrasonic energy source is activated to vibrate the ultrasonic probe in a transverse direction. A transverse ultrasonic vibration of the ultrasonic probe provides a plurality of transverse nodes and transverse anti-nodes along a portion of the longitudinal axis of the ultrasonic probe, causing a cavitation in a medium in communication with the ultrasonic probe in a direction not parallel to the longitudinal axis of the ultrasonic probe to ablate the occlusion.

The following terms and definitions are used herein:

“Ablate” as used herein refers to removing, clearing, destroying or taking away a biological material. “Ablation” as used herein refers to a removal, clearance, destruction, or taking away of the biological material.

“Node” as used herein refers to a region of minimum energy emitted by a probe at or proximal to a specific location along a longitudinal axis of the probe.

“Anti-node” as used herein refers to a region of maximum energy emitted by a probe at or proximal to a specific location along a longitudinal axis of the probe.

“Probe” as used herein refers to a device capable of propagating an energy emitted by the ultrasonic energy source along a longitudinal axis of the probe, resolving this energy into effective cavitational energy at a specific resonance (defined by a plurality of nodes and a plurality of anti-nodes along an “active area” of the probe) and is capable of acoustic impedance transformation of ultrasound energy to mechanical energy. A probe can be a wire.

“Transverse” as used herein refers to vibration of a probe not parallel to the longitudinal axis of the probe. A “transverse wave” as used herein is a wave propagated along a probe in which the direction of the disturbance at each point of the medium is not parallel to the wave vector.

“Biological material” as used herein refers to an aggregation of matter including, but not limited to, a group of similar cells, intravascular blood clots or thrombus, fibrin, calcified plaque, calcium deposits, occlusional deposits, atherosclerotic plaque, fatty deposits, adipose tissues, atherosclerotic cholesterol buildup, fibrous material buildup, arterial stenoses, minerals, high water content tissues, platelets, cellular debris, wastes and other occlusive materials.

“Occlusion” refers to a blockage, a clot, a buildup or a deposit of a matter that results in an obstruction, restriction, obstruction, constriction, blockage or closure at a site of the occlusion.

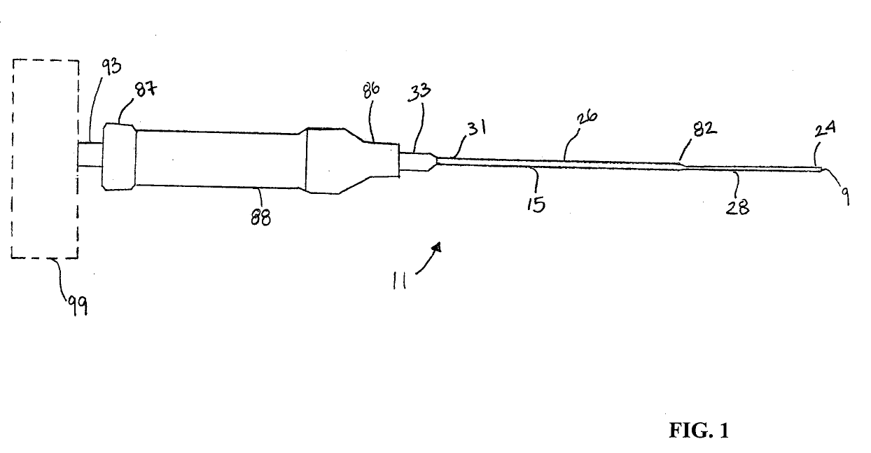

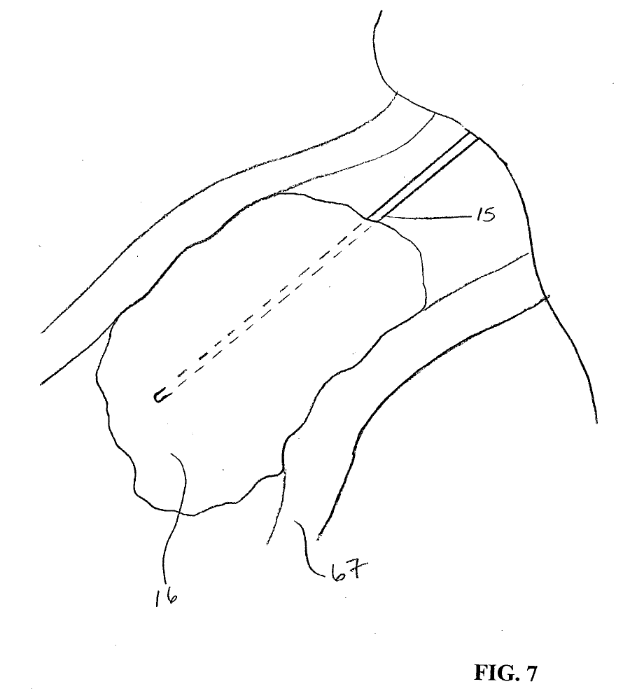

An ultrasonic medical device operating in a transverse mode of the present invention is illustrated generally at 11 in FIG. 1. The ultrasonic medical device 11 includes an ultrasonic probe 15 and an ultrasonic energy source or generator 99 (shown in phantom in FIG. 1 and FIG. 7) for the production of an ultrasonic energy. A handle 88, comprising a proximal end 87 and a distal end 86, surrounds a transducer within the handle 88. The transducer having a first end engaging the ultrasonic energy source 99 and a second end engaging a proximal end 31 of the ultrasonic probe 15 transmits an ultrasonic energy to the ultrasonic probe. A connector 93 engages the ultrasonic energy source 99 to the transducer within the handle 88. The ultrasonic probe 15 includes the proximal end 31, a distal end 24 and a longitudinal axis between the proximal end 31 and the distal end 24. A diameter of the ultrasonic probe 15 decreases from a first defined interval 26 to a second defined interval 28 along the longitudinal axis of the ultrasonic probe 15 over an at least one diameter transition 82. At the distal end 24 of the longitudinal axis of the ultrasonic probe 15, the ultrasonic probe 15 ends in a probe tip 9. A quick attachment-detachment (QAD) system 33 that engages the proximal end 31 of the ultrasonic probe 15 to the transducer within the handle 88 is illustrated generally in FIG. 1. An ultrasonic probe device with a rapid attachment and detachment means is described in the Assignee's co-pending patent applications U.S. Ser. No. 09/975,725; U.S. Ser. No. 10/268,487; U.S. Ser. No. 10/268,843, which further describe the quick attachment-detachment system and the entirety of these applications are hereby incorporated herein by reference.

The ultrasonic probe 15 has a stiffness that gives the ultrasonic probe 15 a flexibility so it can be articulated in the vascular access device. In a preferred embodiment of the present invention, the ultrasonic probe 15 is a wire. In another embodiment of the present invention, the ultrasonic probe 15 is elongated. In a preferred embodiment of the present invention, the diameter of the ultrasonic probe 15 decreases from the first defined interval 26 to the second defined interval 28. In another embodiment of the present invention, the diameter of the ultrasonic probe 15 decreases at greater than two defined intervals. In a preferred embodiment of the present invention, the diameter transitions 82 of the ultrasonic probe 15 are tapered to gradually change the diameter from the proximal end 31 to the distal end 24 along the longitudinal axis of the ultrasonic probe 15. In another embodiment of the present invention, the diameter transitions of the ultrasonic probe 15 are stepwise to change the diameter from the proximal end 31 to the distal end 24 along the longitudinal axis of the ultrasonic probe 15. Those skilled in the art will recognize that there can be any number of defined intervals and diameter transitions, and that the diameter transitions can be of any shape known in the art and be within the spirit and scope of the present invention.

The probe tip 9 can be any shape including, but not limited to, bent, a ball or larger shapes. In one embodiment of the present invention, the ultrasonic energy source 99 is a physical part of the ultrasonic medical device 11. In another embodiment of the present invention, the ultrasonic energy source 99 is not a physical part of the ultrasonic medical device 11.

In a preferred embodiment of the present invention, the cross section of the ultrasonic probe 15 is approximately circular. In other embodiments of the present invention, a shape of the cross section of the ultrasonic probe 15 includes, but is not limited to, square, trapezoidal, oval, triangular, circular with a flat spot and similar cross sections. Those skilled in the art will recognize that other cross sectional geometric configurations known in the art would be within the spirit and scope of the present invention.

The ultrasonic probe 15 is inserted into the vascular access device and may be disposed of after use. In a preferred embodiment of the present invention, the ultrasonic probe 15 is for a single use and on a single patient. In a preferred embodiment of the present invention, the ultrasonic probe 15 is disposable. In another embodiment of the present invention, the ultrasonic probe 15 can be used multiple times.

The amount of cavitation energy to be applied to a particular site requiring treatment is a function of the amplitude and frequency of vibration of the ultrasonic probe 15, the longitudinal length of the ultrasonic probe 15, the geometry at the distal end (24) of the ultrasonic probe 15, the proximity of the ultrasonic probe 15 to the occlusion 16, and the degree to which the length of the ultrasonic probe 15 is exposed to the occlusion 16.

In a preferred embodiment of the present invention, the ultrasonic probe 15 has a small diameter. In a preferred embodiment of the present invention, the diameter of the ultrasonic probe 15 gradually decreases from the proximal end 31 to the distal end 24. In an embodiment of the present invention, the diameter of the distal end 24 of the ultrasonic probe 15 is about 0.004 inches. In another embodiment of the present invention, the diameter of the distal end 24 of the ultrasonic probe 15 is about 0.015 inches. In other embodiments of the present invention, the diameter of the distal end 24 of the ultrasonic probe 15 varies between about 0.003 inches and about 0.025 inches. Those skilled in the art will recognize an ultrasonic probe 15 can have a diameter at the distal end 24 smaller than about 0.003 inches, larger than about 0.025 inches, and between about 0.003 inches and about 0.025 inches and be within the spirit and scope of the present invention.

In an embodiment of the present invention, the diameter of the proximal end 31 of the ultrasonic probe 15 is about 0.012 inches. In another embodiment of the present invention, the diameter of the proximal end 31 of the ultrasonic probe 15 is about 0.025 inches. In other embodiments of the present invention, the diameter of the proximal end 31 of the ultrasonic probe 15 varies between about 0.003 inches and about 0.025 inches. Those skilled in the art will recognize the ultrasonic probe 15 can have a diameter at the proximal end 31 smaller than about 0.003 inches, larger than about 0.025 inches, and between about 0.003 inches and about 0.025 inches and be within the spirit and scope of the present invention.

In an embodiment of the present invention, the diameter of the ultrasonic probe 15 is approximately uniform from the proximal end 31 to the distal end 24 of the ultrasonic probe 15. In another embodiment of the present invention, the diameter of the ultrasonic probe 15 gradually decreases from the proximal end 31 to the distal end 24. In an embodiment of the present invention, the ultrasonic probe 15 may resemble a wire. In an embodiment of the present invention, the gradual change of the diameter from the proximal end 31 to the distal end 24 occurs over the at least one diameter transitions 82 with each diameter transition 82 having an approximately equal length. In another embodiment of the present invention, the gradual change of the diameter from the proximal end 31 to the distal end 24 occurs over a plurality of diameter transitions 82 with each diameter transition 82 having a varying length. The diameter transition 82 refers to a section where the diameter varies from a first diameter to a second diameter.

The length of the ultrasonic probe 15 of the present invention is chosen so as to be resonant in a transverse mode. In an embodiment of the present invention, the ultrasonic probe 15 is between about 30 centimeters and about 300 centimeters in length. In an embodiment of the present invention, the ultrasonic probe (15) is a wire. Those skilled in the art will recognize an ultrasonic probe can have a length shorter than about 30 centimeters and a length longer than about 300 centimeters and be within the spirit and scope of the present invention.

The handle 88 surrounds the transducer located between the proximal end 31 of the ultrasonic probe 15 and the connector 93. In a preferred embodiment of the present invention, the transducer includes, but is not limited to, a horn, an electrode, an insulator, a backnut, a washer, a piezo microphone, and a piezo drive. The transducer converts electrical energy provided by the ultrasonic energy source 99 to mechanical energy. The transducer transmits ultrasonic energy received from the ultrasonic energy source 99 to the ultrasonic probe 15. Energy from the ultrasonic energy source 99 is transmitted along the longitudinal axis of the ultrasonic probe 15, causing the ultrasonic probe 15 to vibrate in a transverse mode. The transducer is capable of engaging the ultrasonic probe 15 at the proximal end 31 with sufficient restraint to form an acoustical mass that can propagate the ultrasonic energy provided by the ultrasonic energy source 99.

The ultrasonic energy source 99 produces a transverse ultrasonic vibration along a portion of the longitudinal axis of the ultrasonic probe 15. The ultrasonic probe 15 can support the transverse ultrasonic vibration along the portion of the longitudinal axis of the ultrasonic probe 15. The transverse mode of vibration of the ultrasonic probe 15 according to the present invention differs from an axial (or longitudinal) mode of vibration disclosed in the prior art. Rather than vibrating in an axial direction, the ultrasonic probe 15 of the present invention vibrates in a direction transverse (not parallel) to the axial direction. As a consequence of the transverse vibration of the ultrasonic probe 15, the occlusion destroying effects of the ultrasonic medical device 11 are not limited to those regions of the ultrasonic probe 15 that may come into contact with the occlusion 16. Rather, as a section of the longitudinal axis of the ultrasonic probe 15 is positioned in proximity to an occlusion, a diseased area or lesion, the occlusion 16 is removed in all areas adjacent to a plurality of energetic transverse nodes and transverse anti-nodes that are produced along a portion of the longitudinal axis of the ultrasonic probe 15, typically in a region having a radius of up to about 6 mm around the ultrasonic probe 15.

Transversely vibrating ultrasonic probes for occlusion ablation are described in the Assignee's co-pending patent applications U.S. Ser. No. 09/776,015; U.S. Ser. No. 09/618,352 and U.S. Ser. No. 09/917,471, which further describe the design parameters for such an ultrasonic probe and its use in ultrasonic devices for an ablation, and the entirety of these applications are hereby incorporated herein by reference.

A vascular introducer used with an ultrasonic probe is described in Assignee's copending patent application U.S. Ser. No. 10/080,787, which further describes the device and its use for clearing debris and the entirety of this application is hereby incorporated herein by reference.

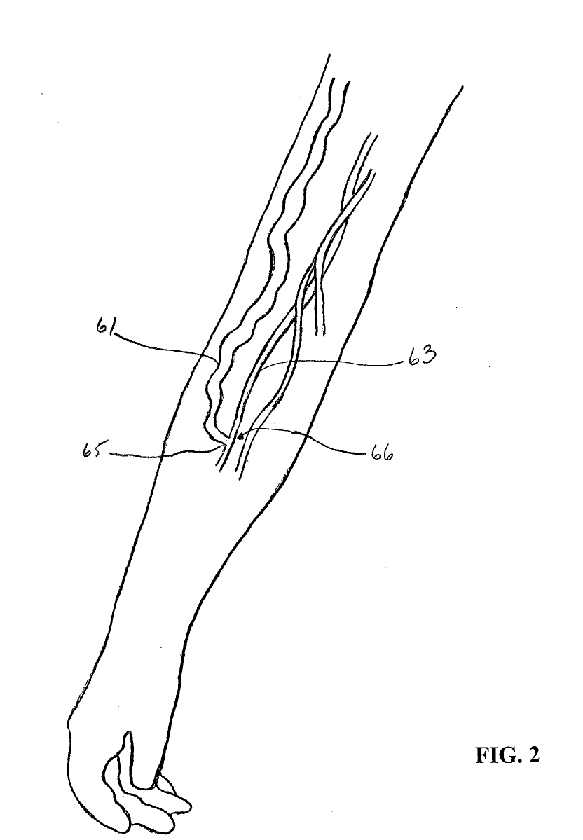

FIG. 2 illustrates an AV fistula 66 formed by engaging an artery 61 to a vein 63 at fistula engagement points 65 in an arm of a patient. The engaging of the artery 61 to the vein 63 provides a permanent access that allows for an increase in a flow of an arterial blood into the vein 63 allowing a percutaneous puncture of the larger and strong vein.

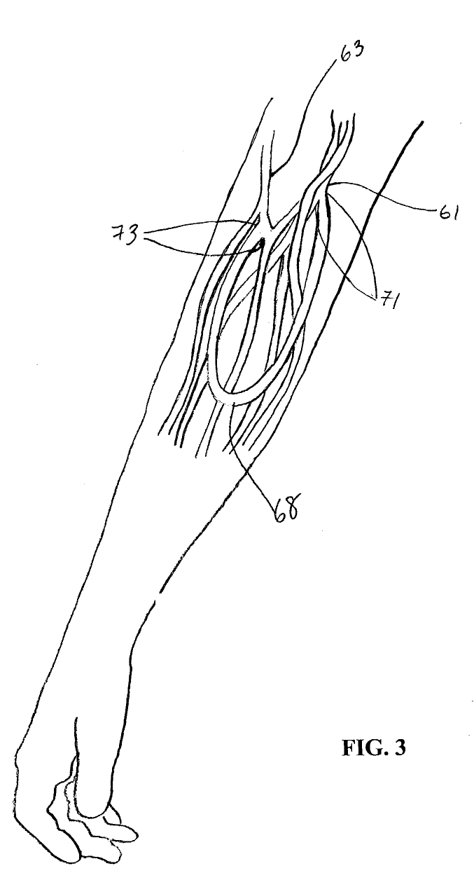

FIG. 3 illustrates a graft 68 formed by engaging an artificial blood vessel to the artery 61 and the vein 63 in the arm of the patient. The graft 68 engages the artery 61 at a graft-artery engagement point 71. The graft 68 engages the vein 63 at a graft-vein engagement point 73.

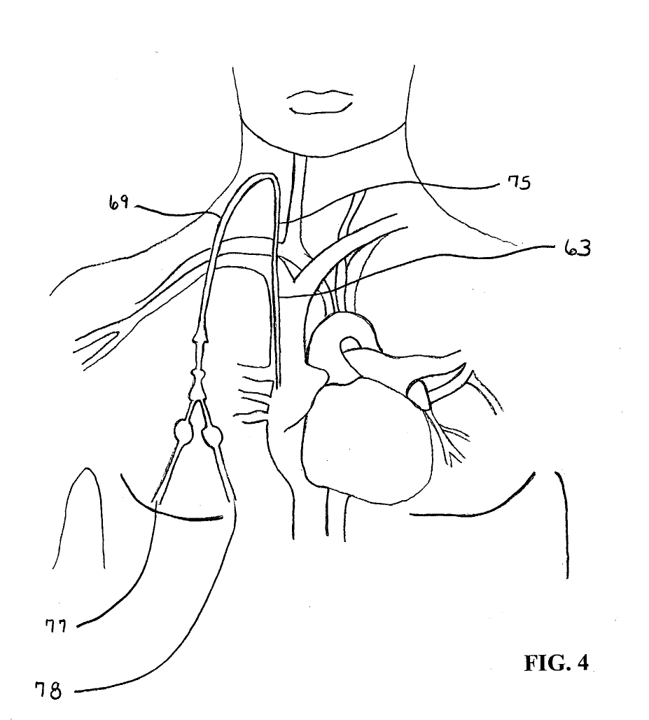

FIG. 4 illustrates a catheter 69 inserted into the vein 63 in a chest region of the patient. The catheter 69 is inserted into the vein 63 at a catheter-vein engagement point 75. The catheter 69 has a catheter outlet access 77 and a catheter inlet access 78 that remove and return blood, respectively, from a machine that treats the blood such as a hemodialysis machine.

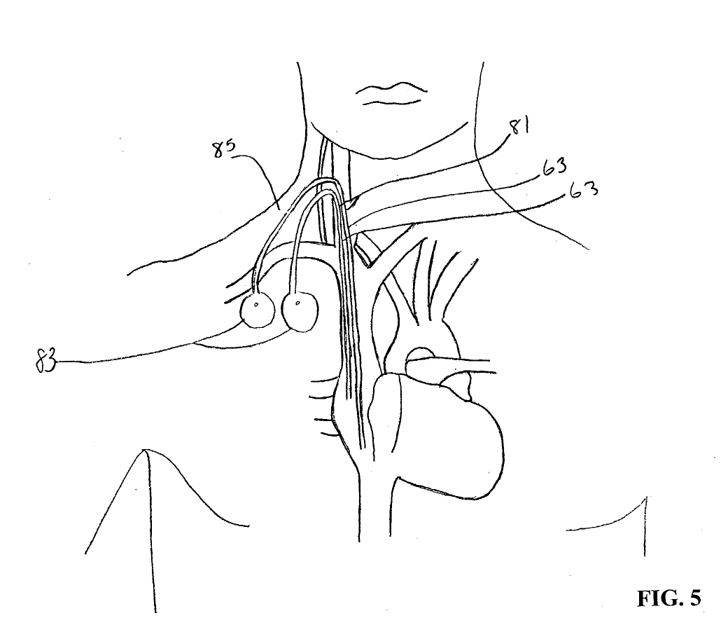

FIG. 5 illustrates a subcutaneous access device 85 comprising a plurality of metallic devices 83 engaging the vein 63 at a subcutaneous access device engagement point 81. The plurality of metallic devices 83 are implanted underneath the skin. The subcutaneous access devices have internal mechanisms that open as a needle is inserted and close when the needle is removed.

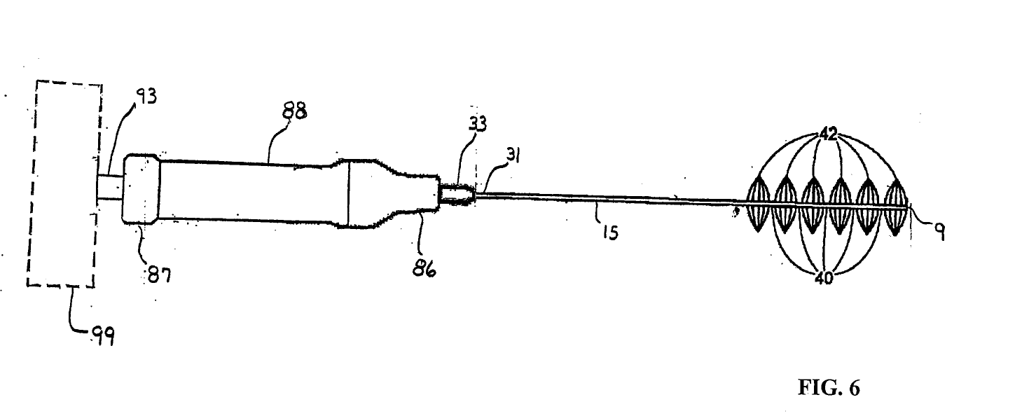

FIG. 6 illustrates an alternative embodiment of the ultrasonic medical device 11 wherein the ultrasonic probe 15 comprises an approximately uniform diameter. The ultrasonic probe 15 comprises a plurality of transverse nodes 40 and transverse anti-nodes 42 at repeating intervals along a portion of the longitudinal axis of the ultrasonic probe 15. The transverse ultrasonic vibration produces the plurality of transverse nodes 40 and transverse anti-nodes 42 along the portion of the longitudinal axis of the ultrasonic probe 15. The transverse nodes 40 are areas of a minimum energy and a minimum vibration. A plurality of transverse anti-nodes 42, or areas of a maximum energy and a maximum vibration, also occur at repeating intervals along the portion of the longitudinal axis of the ultrasonic probe 15. The number of transverse nodes 40 and the transverse anti-nodes 42, and the spacing of the transverse nodes 40 and transverse anti-nodes 42 of the ultrasonic probe 15 depend on the frequency of the energy produced by the ultrasonic energy source 99. The separation of the transverse nodes 40 and the transverse anti-nodes 42 is a function of the frequency, and can be affected by tuning the ultrasonic probe 15. In a properly tuned ultrasonic probe 15, the transverse anti-nodes 42 will be found at a position exactly one-half of the distance between the transverse nodes 40 located adjacent to each side of the transverse anti-nodes 42. A length and the cross section of the ultrasonic probe 15 are sized to support the transverse ultrasonic vibration with a plurality of transverse nodes 40 and transverse anti-nodes 42 along the portion of the longitudinal axis of the ultrasonic probe 15. In a preferred embodiment of the present invention, more than one of the plurality of transverse anti-nodes are in communication with the occlusion 16.

The effects of the ultrasonic medical device 11 operating in a transverse mode of the present invention for destroying the material comprising the occlusion 16 are not limited to those regions of the probe 15 that may come into contact with the occlusion 16. Rather, as the segment of the longitudinal axis of the ultrasonic probe 15 is moved through an area of the occlusion 16, the occlusion 16 is removed in all areas adjacent to the plurality of transverse anti-nodes 42 being produced along a portion of the longitudinal axis of the ultrasonic probe 15. The extent of the cavitational energy produced by the ultrasonic probe 15 is such that the cavitational energy extends radially outward from the longitudinal axis of the ultrasonic probe 15 at the transverse anti-nodes 42 along the portion of the longitudinal axis of the ultrasonic probe 15. In this way, actual treatment time using the transverse mode ultrasonic medical device 11 according to the present invention is greatly reduced as compared to methods disclosed in the prior art that primarily utilize longitudinal vibration (along the axis of the ultrasonic probe) for ablation of the occlusion. Utilizing longitudinal vibration limits treatment to the tip of the probe in prior art devices.

By eliminating the axial motion of the ultrasonic probe 15 and allowing transverse vibrations only, the active ultrasonic probe 15 can cause fragmentation of large areas of the material comprising the occlusion 16 that span the length of the active area of the ultrasonic probe 15 due to generation of multiple cavitational transverse anti-nodes 42 along the longitudinal axis of the ultrasonic probe 15 not parallel to the longitudinal axis of the ultrasonic probe 15. Since substantially larger affected areas can be denuded of the occlusion 16 in a short time, actual treatment time using the transverse mode ultrasonic medical device 11 according to the present invention is greatly reduced as compared to methods using prior art probes that primarily utilize longitudinal vibration (along the axis of the probe) for ablation. A distinguishing feature of the present invention is the ability to utilize ultrasonic probes 15 of extremely small diameter compared to prior art probes, without loss of efficiency, because the occlusion fragmentation process is not dependent on the area of the probe tip 9. Highly flexible ultrasonic probes 15 can therefore be designed to mimic device shapes that enable facile insertion into occlusion 16 spaces or extremely narrow interstices that contain the material comprising the occlusion 16. Another advantage provided by the present invention is the ability to rapidly remove the material comprising the occlusion 16 from large areas within cylindrical or tubular surfaces.

A significant advantage of the present invention is that the ultrasonic medical device 11 physically destroys and removes the material comprising the occlusion 16 (especially adipose or other high water content tissue) through the mechanism of non-thermal cavitation. Cavitation is a process in which small voids are formed in a surrounding fluid through the rapid motion of the ultrasonic probe 15 and the voids are subsequently forced to compress. The compression of the voids creates a wave of acoustic energy which acts to dissolve the matrix binding together the occlusion 16, while having no damaging effects on healthy tissue. The ultrasonic energy source 99 provides a low power electric signal of approximately 2 watts to the transducer, which then transforms the electric signal into acoustic energy. Longitudinal motion created within the transducer is converted into a standing transverse wave along the portion of the longitudinal axis of the ultrasonic probe 15, which generates acoustic energy in the surrounding medium through cavitation. The acoustic energy dissolves the matrix-of the occlusion 16. In a preferred embodiment of the present invention, the occlusion 16 comprises a biological material. The transverse anti-nodes 42 cause a cavitation in a medium in communication with the ultrasonic probe 15 in a direction not parallel to the longitudinal axis of the ultrasonic probe 15. In a preferred embodiment of the present invention, more than one of the plurality of transverse anti-nodes 42 are in communication with the occlusion 16.

FIG. 7 illustrates a segment of the longitudinal axis of the ultrasonic probe 15 inserted into the vascular access device 67 and engaging an occlusion 16 in the vascular access device 67. As previously stated, the vascular access device 67 may be the fistula 66, the graft 68, the catheter 69 or the subcutaneous access device 85. Those skilled in the art will recognize there are other vascular access devices known in the art that are within the spirit and scope of the present invention.

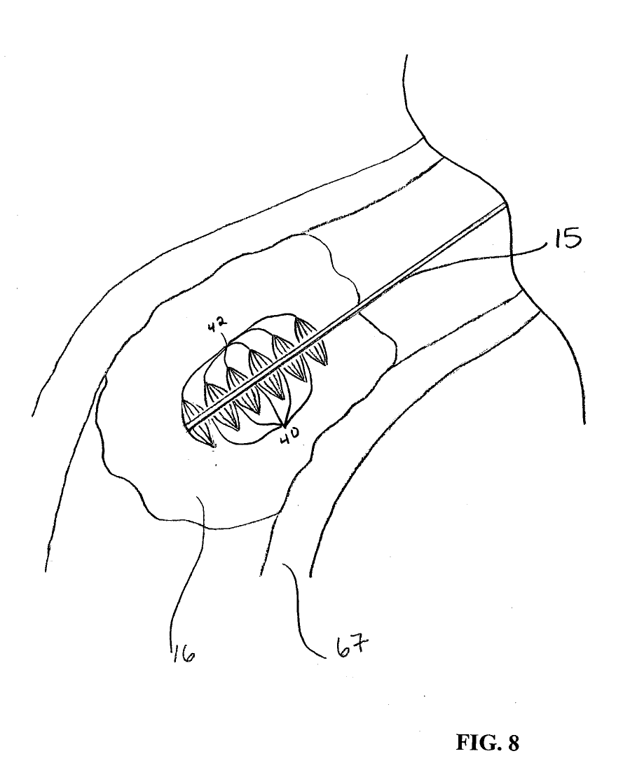

FIG. 8 shows a section of the longitudinal axis of the ultrasonic probe 15 treating the occlusion 16 within the vascular access device 67 after a short timeframe in which the ultrasonic energy source is activated. In FIG. 8, a portion of the occlusion 16 is removed. The ultrasonic energy produced by the ultrasonic probe 15 is in the form of very intense, high frequency sound vibrations that result in physical reactions in the water molecules within a body tissue or surrounding fluids in proximity to the ultrasonic probe 15. These reactions ultimately result in a process called “cavitation,” which can be thought of as a form of cold (i.e., non-thermal) boiling of the water in the body tissue, such that microscopic voids are rapidly created and destroyed in the water creating cavities in their wake. As surrounding water molecules rush in to fill the cavity created by the collapsed voids, they collide with each other with great force. Cavitation results in shock waves running outward from the collapsed voids which can wear away or destroy material such as surrounding tissue in the vicinity of the ultrasonic probe 15. The process of cavitation removes large volumes of material comprising the occlusion 16 in the vascular access device 67, decreasing the size of the occlusion 16 as shown in FIG. 8.

The removal of the occlusion 16 by cavitation also provides the ability to remove large volumes of material comprising the occlusion 16 with the small diameter ultrasonic probe 15, while not affecting healthy tissue. The use of cavitation as the mechanism for destroying the occlusion 16 allows the present invention to destroy and remove the material comprising the occlusion 16 within a range of temperatures of about ±7° C. from normal body temperature. Therefore, complications attendant with the use of thermal destruction or necrosis, such as swelling or edema, as well as loss of elasticity are avoided.

The number of transverse nodes 40 and transverse anti-nodes 42 occurring along the longitudinal axis of the ultrasonic probe 15 is modulated by changing the frequency of energy supplied by the ultrasonic energy source 99. The exact frequency, however, is not critical and the ultrasonic energy source 99 run at, for example, about 20 kHz is sufficient to create an effective number of occlusion 16 destroying transverse anti-nodes 42 along the longitudinal axis of the ultrasonic probe 15. The low frequency requirement of the present invention is a further advantage in that the low frequency requirement leads to less damage to healthy tissue. Those skilled in the art understand it is possible to adjust the dimensions of the ultrasonic probe 15, including diameter, length and distance to the ultrasonic energy source 99, in order to affect the number and spacing of the transverse nodes 40 and transverse anti-nodes 42 along a portion of the longitudinal axis of the ultrasonic probe 15.

The present invention allows the use of ultrasonic energy to be applied to the occlusion 16 selectively, because the ultrasonic probe 15 conducts energy across a frequency range from about 20 kHz through about 80 kHz. The amount of ultrasonic energy to be applied to a particular treatment site is a function of the amplitude and frequency of vibration of the ultrasonic probe 15. In general, the amplitude or throw rate of the energy is in the range of about 25 microns to about 250 microns, and the frequency in the range of about 20 kHz to about 80 kHz. In a preferred embodiment of the present invention, the frequency of ultrasonic energy is from about 20 kHz to about 35 kHz. Frequencies in this range are specifically destructive of occlusions 16 including, but not limited to, hydrated (water-laden) tissues such as endothelial tissues, while substantially ineffective toward high-collagen connective tissue, or other fibrous tissues including, but not limited to, vascular tissues, epidermal, or muscle tissues.

In a preferred embodiment of the present invention, the transducer transmits ultrasonic energy from the ultrasonic energy source 99 to the longitudinal axis of the ultrasonic probe 15 to oscillate the ultrasonic probe 15 in a direction transverse to its longitudinal axis. In a preferred embodiment of the present invention, the transducer is a piezoelectric transducer that is coupled to the ultrasonic probe 15 to enable transfer of ultrasonic excitation energy and cause the ultrasonic probe 15 to oscillate in the transverse direction relative to the longitudinal axis. In an alternative embodiment of the present invention, a magneto-strictive transducer may be used for transmission of the ultrasonic energy. The ultrasonic probe 15 is designed to have the cross section with a small profile, which also allows the ultrasonic probe 15 to flex along its length, thereby allowing the ultrasonic probe 15 to be used in a minimally invasive manner. A significant feature of the present invention resulting from the transversely generated energy is the retrograde movement of biological material, e.g., away from the probe tip 9 and along the longitudinal axis of the ultrasonic probe 15.

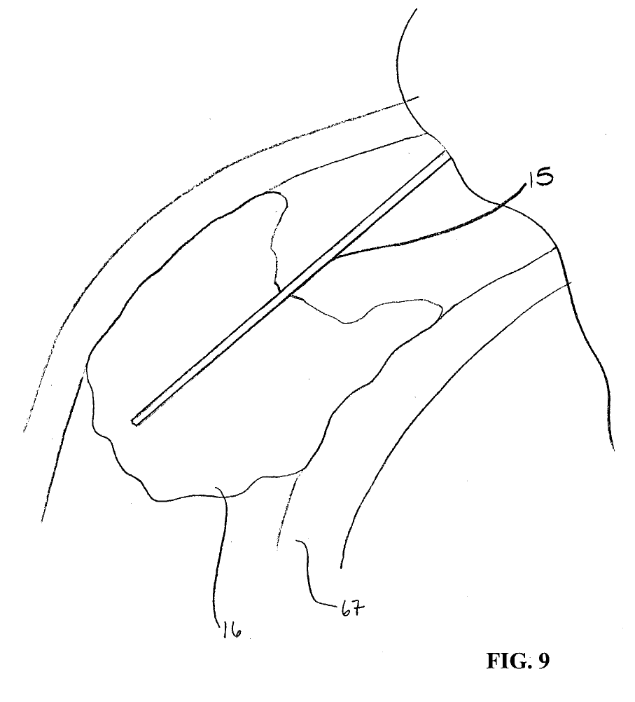

FIG. 9 shows the ultrasonic probe 15 in proximity to the occlusion 16 wherein only a small amount of the occlusion 16 remains. The progressive ablation of the occlusion 16 continues with an additional removal of the occlusion 16 from within the vascular access device 67 as shown in FIG. 9.

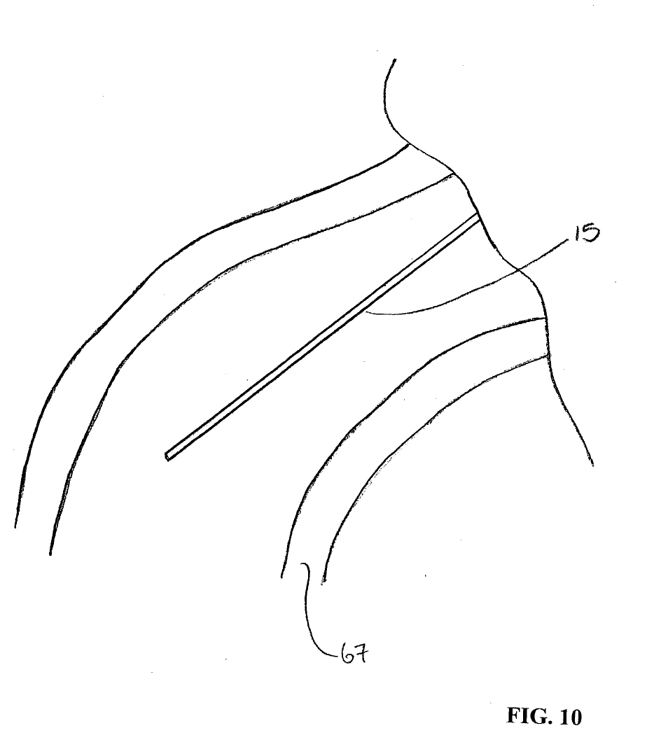

FIG. 10 shows the complete resolution of the occlusion 16 in the vascular access device 67 in which the occlusion 16 in the vascular access device 67 is completely ablated. After removal of the occlusion 16 from the vascular access device 67 using the ultrasonic medical device 11 of the present invention, normal blood flow is restored in the vascular access device 67 and downstream.

The present invention provides a method of removing an occlusion 16 in a vascular access device 67. The section of the longitudinal axis of the ultrasonic probe 15 engages the occlusion 16 in the vascular access device 67. The ultrasonic probe 15 is inserted into the vascular access device 67 and the ultrasonic energy source 99 is activated, producing an ultrasonic energy to vibrate the ultrasonic probe 15 in a transverse direction, thereby providing a plurality of transverse anti-nodes 42 along a portion of the longitudinal axis of the ultrasonic probe 15. The transverse anti-nodes 42 cause a cavitation in a medium in communication with the ultrasonic probe 15 to ablate the occlusion 16.

The present invention provides a method of ablating an occlusion 16 in a vascular access device 67 with the ultrasonic medical device 11. In an embodiment of the present invention, the vascular access device 67 is the graft 68. In another embodiment of the present invention, the vascular access device 67 is the fistula 66. In another embodiment of the present invention, the vascular access device 67 is the catheter 69. In another embodiment of the present invention, the vascular access device 67 is the subcutaneous access device 85. In an embodiment of the present invention, the segment of the longitudinal axis of the ultrasonic probe 15 is moved within the vascular access device 67 and the ultrasonic energy source 99 is activated. In an embodiment of the present invention, the ultrasonic probe 15 is rotated along the occlusion 16 within the vascular access device 67. In another embodiment of the present invention, the ultrasonic probe 15 is swept along the occlusion 16 within the vascular access device 67. In another embodiment of the present invention, the ultrasonic probe 15 is moved back and forth along the occlusion 16 within the vascular access device 67. Those skilled in the art will recognize the segment of the longitudinal axis of the ultrasonic probe can be moved within the vascular access device in many ways and be within the spirit and scope of the present invention.

The present invention provides a method of effectively removing an occlusion 16 in a vascular access device 67 to prevent complications in procedures such as treating blood. The present invention is used to remove occlusions 16 in vascular access devices 67 including fistulas, grafts, catheters, subcutaneous access devices and other similar devices. The present invention provides a method of effectively removing the occlusion 16 from the vascular access device 67 that is simple, user-friendly, effective, reliable and cost effective.

All patents, patent applications, and published references cited herein are hereby incorporated herein by reference in their entirety. While this invention has been particularly shown and described with references to preferred embodiments thereof, it will be understood by those skilled in the art that various changes in form and details may be made therein without departing from the scope of the invention encompassed by the appended claims.

What is claimed is:

1. An ultrasonic medical device for ablating an occlusion in a vascular access device comprising:
an ultrasonic probe having a proximal end, a distal end and a longitudinal axis therebetween;
an ultrasonic energy source that produces an ultrasonic energy; and
a transducer for transferring the ultrasonic energy from the ultrasonic energy source to the ultrasonic probe, the transducer having a first end engaging the ultrasonic energy source and a second end engaging the proximal end of the ultrasonic probe,
wherein the ultrasonic energy source produces a transverse ultrasonic vibration along the longitudinal axis of the ultrasonic probe to ablate the occlusion in the vascular access device.

2. The device of claim 1 wherein the vascular access device is selected from a group consisting of a graft, a fistula, a catheter and a subcutaneous access device.

3. The device of claim 1 wherein a length and a cross section of the ultrasonic probe are sized to support the transverse ultrasonic vibration with a plurality of transverse nodes and transverse anti-nodes along a portion of the longitudinal axis of the ultrasonic probe.

4. The device of claim 3 wherein the transverse anti-nodes are points of a maximum transverse energy along the portion of the longitudinal axis of the ultrasonic probe.

5. The device of claim 3 wherein the transverse anti-nodes cause a cavitation in a medium in communication with the ultrasonic probe.

6. The device of claim 3 wherein more than one of the plurality of transverse anti-nodes are in communication with the occlusion.

7. The device of claim 1 wherein the ultrasonic probe is for a single use on a single patient.

8. The device of claim 1 wherein the ultrasonic probe is disposable.

9. The device of claim 1 wherein the occlusion comprises a biological material.

10. An elongated flexible probe for removing an occlusion in a vascular access device comprising:
a proximal end, a distal end and a longitudinal axis therebetween,
wherein the elongated flexible probe supports a transverse ultrasonic vibration along a portion of the longitudinal axis of the elongated flexible probe to remove the occlusion.

11. The device of claim 10 wherein the vascular access device is selected from a group consisting of a graft, a fistula, a catheter and a subcutaneous access device.

12. The device of claim 10 wherein the elongated flexible probe is a wire.

13. The device of claim 10 wherein the elongated flexible probe has a stiffness that gives the elongated flexible probe a flexibility to be articulated in the vascular access device.

14. The device of claim 10 wherein an ultrasonic energy source produces the transverse ultrasonic vibration along the portion of the longitudinal axis of the elongated flexible probe.

15. The device of claim 10 wherein the transverse ultrasonic vibration of the elongated flexible probe provides a plurality of transverse anti-nodes along the portion of the longitudinal axis of the elongated flexible probe.

16. The device of claim 15 wherein the transverse anti-nodes are points of a maximum transverse energy along the portion of the longitudinal axis of the elongated flexible probe.

17. The device of claim 15 wherein the transverse anti-nodes cause a cavitation in a medium in communication with the elongated flexible probe in a direction not parallel to the longitudinal axis of the elongated flexible probe.

18. The device of claim 10 wherein a cross section of the elongated flexible probe has a small profile.

19. The device of claim 10 wherein a diameter of the elongated flexible probe is approximately uniform along the longitudinal axis of the elongated flexible probe.

20. The device of claim 10 wherein a diameter of the elongated flexible probe varies from the proximal end of the elongated flexible probe to the distal end of the elongated flexible probe.

21. The device of claim 10 wherein the occlusion comprises a biological material.

22. A method of removing an occlusion in a vascular access device comprising:
inserting an elongated ultrasonic probe into the vascular access device; and
activating an ultrasonic energy source, wherein the ultrasonic energy source provides an ultrasonic energy to produce a transverse ultrasonic vibration in the elongated ultrasonic probe to remove the occlusion in the vascular access device.

23. The method of claim 22 wherein the vascular access device is selected from a group consisting of a graft, a fistula, a catheter, and a subcutaneous access device.

24. The method of claim 22 wherein a segment of a longitudinal axis of the elongated ultrasonic probe is inserted into the vascular access device.

25. The method of claim 22 wherein the ultrasonic energy source produces the transverse ultrasonic vibration along a portion of a longitudinal axis of the elongated ultrasonic probe.

26. The method of claim 22 wherein the transverse ultrasonic vibration of the elongated ultrasonic probe provides a plurality of transverse anti-nodes along a portion of a longitudinal axis of the elongated ultrasonic probe.

27. The method of claim 26 wherein the transverse anti-nodes are points of a maximum transverse energy along the portion of the longitudinal axis of the elongated ultrasonic probe.

28. The method of claim 26 wherein the transverse anti-nodes cause a cavitation in a medium in communication with the elongated ultrasonic probe in a direction not parallel to the longitudinal axis of the elongated ultrasonic probe.

29. The method of claim 26 wherein more than one of the plurality of transverse antinodes are in communication with the occlusion.

30. The method of claim 22 wherein a length and a cross section of the elongated ultrasonic probe are sized to support the transverse ultrasonic vibration with a plurality of transverse nodes and transverse anti-nodes along a portion of a longitudinal axis of the elongated ultrasonic probe.

31. The method of claim 22 wherein the elongated ultrasonic probe can support the transverse ultrasonic vibration along a portion of a longitudinal axis of the elongated ultrasonic probe to remove the occlusion.

32. The method of claim 22 wherein a first end of a transducer engages the ultrasonic energy source and a second end of the transducer engages a proximal end of the elongated ultrasonic probe.

33. The method of claim 22 wherein a diameter of the elongated ultrasonic probe is approximately uniform along a longitudinal axis of the elongated ultrasonic probe.

34. The method of claim 22 wherein a diameter of the elongated ultrasonic probe varies from a proximal end of the elongated ultrasonic probe to a distal end of the elongated ultrasonic probe.

35. The method of claim 22 wherein a cross section of the elongated ultrasonic probe has a small profile.

36. The method of claim 22 wherein the elongated ultrasonic probe is for a single use on a single patient.

37. The method of claim 22 wherein the elongated ultrasonic probe is disposable.

38. The method of claim 22 wherein the elongated ultrasonic probe has a stiffness that gives the elongated ultrasonic probe a flexibility to be articulated in the vascular access device.

39. The method of claim 22 wherein the occlusion comprises a biological material.

40. A method of ablating an occlusion located in a vascular access device comprising:
inserting a segment of a longitudinal axis of a flexible ultrasonic probe into the vascular access device;
activating an ultrasonic energy source to produce a transverse ultrasonic vibration along the longitudinal axis of the flexible ultrasonic probe; and
moving the segment of the longitudinal axis of the flexible ultrasonic probe within the vascular access device to ablate the occlusion in the vascular access device.

41. The method of claim 40 wherein the longitudinal axis of the flexible ultrasonic probe is rotated within the vascular access device.

42. The method of claim 40 wherein the flexible ultrasonic probe is swept along the occlusion within the vascular access device.

43. The method of claim 40 wherein the flexible ultrasonic probe is moved back and forth along the occlusion within the vascular access device.

44. The method of claim 40 wherein the vascular access device is selected from a group consisting of a graft, a fistula, a catheter, and a subcutaneous access device.

45. The method of claim 40 wherein a transducer transmits an ultrasonic energy to the flexible ultrasonic probe causing a plurality of transverse ultrasonic vibrations along the longitudinal axis of the flexible ultrasonic probe.

46. The method of claim 40 wherein the transverse ultrasonic vibration of the flexible ultrasonic probe provides a plurality of transverse anti-nodes along a portion of the longitudinal axis of the flexible ultrasonic probe.

47. The method of claim 46 wherein the transverse anti-nodes are points of a maximum transverse energy along the portion of the longitudinal axis of the flexible ultrasonic probe.

48. The method of claim 46 wherein the transverse anti-nodes cause a cavitation in a medium in communication with the flexible ultrasonic probe.

49. The method of claim 46 wherein more than one of the plurality of transverse antinodes are in communication with the occlusion.

50. The method of claim 40 wherein a length and a cross section of the flexible ultrasonic probe are sized to support the transverse ultrasonic vibration with a plurality of transverse nodes and transverse anti-nodes along a portion of the longitudinal axis of the flexible ultrasonic probe.

51. The method of claim 40 wherein a first end of a transducer engages the ultrasonic energy source and a second end of the transducer engages a proximal end of the flexible ultrasonic probe.
